(19) United States
(12) Patent Application Publication
(10) Pub. No.: US 2020/0188012 A1
(43) Pub. Date: Jun. 18, 2020

(54) JAW MEMBERS OF ELECTROSURGICAL INSTRUMENTS AND METHODS OF MANUFACTURE THEREOF

(71) Applicant: COVIDIEN LP, MANSFIELD, MA (US)

(72) Inventors: MONTE S. FRY, LONGMONT, CO (US); JEFFREY M. ROY, BOULDER, CO (US)

(21) Appl. No.: 16/221,928

(22) Filed: Dec. 17, 2018

(51) Int. Cl. A61B 18/14

(52) U.S. Cl. CPC A61B 18/1445; A61B 2018/0063

(57) ABSTRACT

A jaw member of an electrosurgical forceps includes a jaw body, an electrically conductive tissue sealing surface extending along a length of the jaw body, and an insulative housing coupled to the jaw body and the sealing surface. The insulative housing has a proximal end defining an oblique surface.

### FIELD

The present disclosure relates generally to the field of surgical instruments. In particular, the disclosure relates to jaw members of endoscopic electrosurgical forceps.

### BACKGROUND

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, the pressure applied to the vessel and the gap distance established between the electrodes are controlled.

### SUMMARY

The techniques of this disclosure generally relate to a jaw member of an electrosurgical instrument having a jaw overmold housing with an oblique proximal end for facilitating entry and exit of the jaw member from a trocar. The jaw member may also have a plurality of anchor holes formed in the jaw body for improving the fixation of the jaw overmold housing on the jaw body.

In one aspect of the present disclosure, provided is a jaw member of an electrosurgical forceps including a jaw body, an electrically conductive tissue sealing surface extending along a length of the jaw body, and an insulative housing coupling the jaw body and the sealing surface. The sealing surface is configured to connect to a source of electrosurgical energy for conducting electrosurgical energy through tissue. The insulative housing has a proximal end defining an oblique surface.

In aspects, the oblique surface may terminate proximally at a proximal edge of the insulative housing.

In some aspects, the proximal edge of the insulative housing may extend perpendicularly relative to a longitudinal plane defined by the electrically conductive tissue sealing surface.

In further aspects, the oblique surface may extend downwardly at an angle from the proximal edge of the insulative housing to a bottom surface of the insulative housing.

In other aspects, the oblique surface may be non-parallel relative to a longitudinal plane defined by the electrically conductive tissue sealing surface and non-perpendicular relative to the longitudinal plane.

In aspects, the oblique surface may be a pair of oblique surfaces each disposed on a respective lateral side of the insulative housing.

In some aspects, the insulative housing may be overmolded to the jaw body and the electrically conductive tissue sealing surface.

In further aspects, the jaw body may have a pair of lateral sides. The insulative housing may be overmolded to the pair of lateral sides.

In other aspects, each of the pair of lateral sides of the jaw body may define a plurality of holes for receiving a portion of the insulative housing therein.

In aspects, one of the holes may disposed adjacent the oblique surface of the insulative housing.

In some aspects, the jaw member may further include a pair of laterally spaced parallel flanges extending proximally from a proximal end of the jaw body. Two of the holes may be formed in the respective pair of flanges.

In further aspects, the oblique surface may be a pair of oblique surfaces each having at least a portion disposed over the respective pair of flanges.

In other aspects, the holes may be three holes.

In aspects, the jaw member may further include a jaw insert disposed within the jaw body and electrically insulating the electrically conductive tissue sealing surface from the jaw body.

As is traditional, the term “distal” refers herein to an end of the electrosurgical instrument or component thereof that is farther from an operator, and the term “proximal” refers herein to the end of the electrosurgical forceps or component thereof that is closer to the operator.

### BRIEF DESCRIPTION OF THE DRAWINGS

Objects and features of the present disclosure will become apparent to those of ordinary skill in the art when descriptions of various embodiments thereof are read with reference to the accompanying drawings, of which:

FIG. 1 is a perspective view of an electrosurgical forceps according to an embodiment of the present disclosure including a housing, an elongated shaft, and an end effector;

FIG. 2A is a side, perspective view of the end effector of FIG. 1 illustrated in an open configuration;

FIG. 2B is a side, perspective view of the end effector of FIG. 1 illustrated in a closed configuration;

FIG. 3 is a transverse, cross-sectional view taken through a lower jaw member of the end effector shown in FIG. 2A;

FIG. 4 is a side, perspective view illustrating a jaw body of the lower jaw member of FIG. 3;

FIG. 5 is a side view of the jaw body of FIG. 4;

FIG. 6 is a side view of the jaw member of FIG. 3; and

FIG. 7 is a bottom view of the jaw member of FIG. 6.

### DETAILED DESCRIPTION

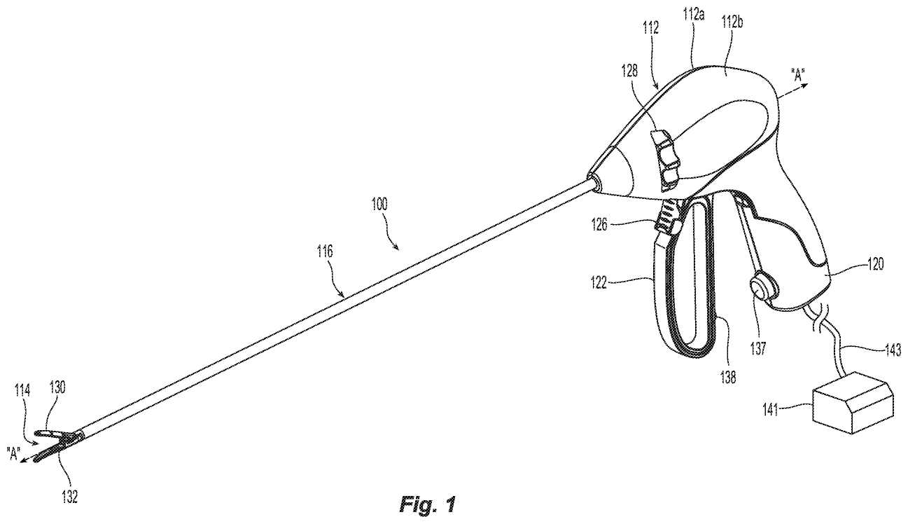

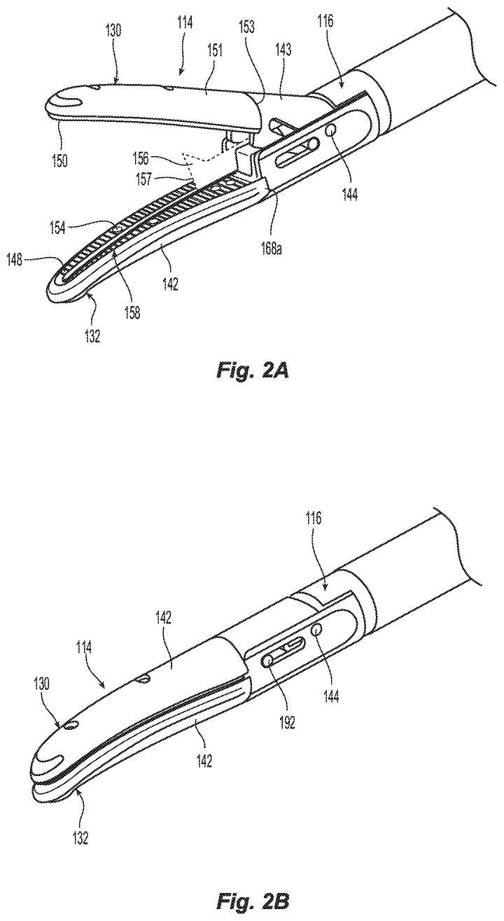

Referring initially to FIGS. 1, 2A, and 2B, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding, mechanical fastening, or other suitable assembly methods.

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein upper and lower jaw members 130, 132 are disposed in spaced relation to one another, and a closed or clamping configuration (FIG. 2B) wherein the jaw members 130, 132 are closer together. Approximation of the movable handle 122 with the stationary handle 120 moves the end effector 114 to the closed configuration and separation of the movable handle 122 from the stationary handle 120 moves the end effector 114 to the open configuration. The trigger 126 is operable to extend and retract a knife blade 156 (FIG. 2A) through the end effector 114 when the end effector 114 is in the closed configuration. The rotation knob 128 rotates the elongated shaft 116 and the end effector 114 about a longitudinal axis A-A extending through the forceps 100.

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch (not shown) disposed within the stationary handle 120 and is engageable by a button activation post 138 extending from a proximal side of the moveable handle 122 upon proximal movement of the moveable handle 122 to an actuated or proximal position. The switch is in electrical communication with an electrosurgical generator 141 via suitable electrical wiring (not explicitly shown) extending from the housing 112 through a cable 143 extending between the housing 112 and the electrosurgical generator 141. The cable 143 may include a connector (not shown) thereon such that the forceps 100 may be selectively coupled electrically to the generator 141.

The end effector 114 may be moved from the open configuration, in which tissue (not shown) is received between the jaw members 130, 132, and the closed configuration, in which the tissue is clamped for subsequent treatment. The jaw members 130, 132 pivot about a pivot pin 144 to move the end effector 114 to the closed configuration wherein sealing plates 148, 150 of the jaw members 132, 130 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 associated with, e.g., disposed on or adjacent, the sealing plates 148, 150.

The upper and lower jaw members 130, 132 are electrically coupled to the cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (−) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141.

Electrosurgical energy may be delivered to the tissue through the electrically conductive seal plates 148, 150 to affect a tissue seal. Once a tissue seal is established, the knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is inhibited.

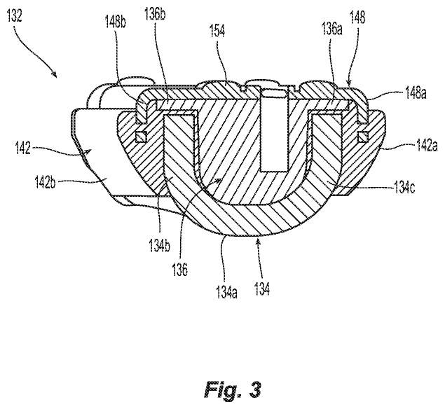

With reference to FIGS. 3-5, the lower jaw member 132 is constructed of four major components: a jaw body 134, a jaw insert 136, the sealing plate 148, and an insulative housing 142. The jaw body 134 may be a metal, such as, for example, stainless steel, and is configured to support the remaining components of the jaw member 132. The jaw body 134 has a generally U-shaped transverse cross-sectional profile and includes a bottom portion 134a and a pair of lateral sides 134b, 134c extending upwardly from the bottom portion 134a. The lateral sides 134b, 134c of the jaw body 134 each define one or more, e.g., two, holes 146a, 146b therethrough. The holes 146a, 146b are longitudinally spaced from one another along a longitudinal axis of the jaw member 132. In some aspects, the holes 146a 146b may be configured as depressions in the lateral sides 134b, 134c.

The lower jaw member 132 has a pair of laterally-spaced parallel flanges 160, 162 extending proximally from a proximal end 164 of the jaw body 134. In embodiments, the flanges 160, 162 and the jaw body 134 may be monolithically formed or integrally connected. The flanges 160, 162 are coupled to a distal end of the shaft 116 (FIG. 2A) and have a proximal flange portion of the upper jaw 130 pivotably coupled thereto. Each of the flanges 160, 162 also defines a hole 146c therethrough that, in embodiments, may be disposed in alignment with the respective pair of holes 146a, 146b in the jaw body 134. The holes 146a, 146b, 146c are each configured to receive a corresponding protuberance (not shown) of the insulative housing 142 to facilitate anchoring of the insulative housing 142 to the jaw body 134, e.g., during overmolding of the insulative housing 142 about the jaw body 134. The holes 146a-c may assume any suitable shape, such as, for example, rounded, squared, flat, or the like. In some aspects, each of the flanges 160, 162 may have a depression formed therein instead of the hole 146c.

The jaw insert 136 of the lower jaw member 132 is fabricated from an insulative material and is received within a cavity 166 defined in the jaw body 134. The jaw insert 136 may be constructed of an electrically insulative plastic such as a polyphthalamide (PPA) (e.g., Amodel®), polycarbonate (PC), acrylonitrile butadiene styrene (ABS), a blend of PC and ABS, nylon, ceramic, etc. The jaw insert 136 has a pair of opposing laterally-extending sides 136a, 136b. The sides 136a, 136b of the jaw insert 136 are supported on the respective lateral sides 134b, 134c of the jaw body 134. In embodiments, the jaw insert 136 is overmolded.

The seal plate 148 is supported on the jaw insert 136, whereby the jaw insert 136 electrically isolates the seal plate 148 from the jaw body 134. As mentioned above, the seal plate 148 is configured to connect to a source of electrosurgical energy, such as, for example, electrosurgical generator 141 (FIG. 1) for conducting the electrosurgical energy through tissue grasped between the jaw members 130, 132. The seal plate 148 and the jaw insert 136 collectively define the knife channel 158, which is dimensioned for passage of the knife 156 (FIG. 2A) of the electrosurgical forceps 100. The seal plate 148 has a pair of lateral sides 148a, 148b curved or otherwise bent down and around both the lateral sides 136a, 136b of the jaw insert 136 and the lateral sides 134a, 134b of the jaw body 134.

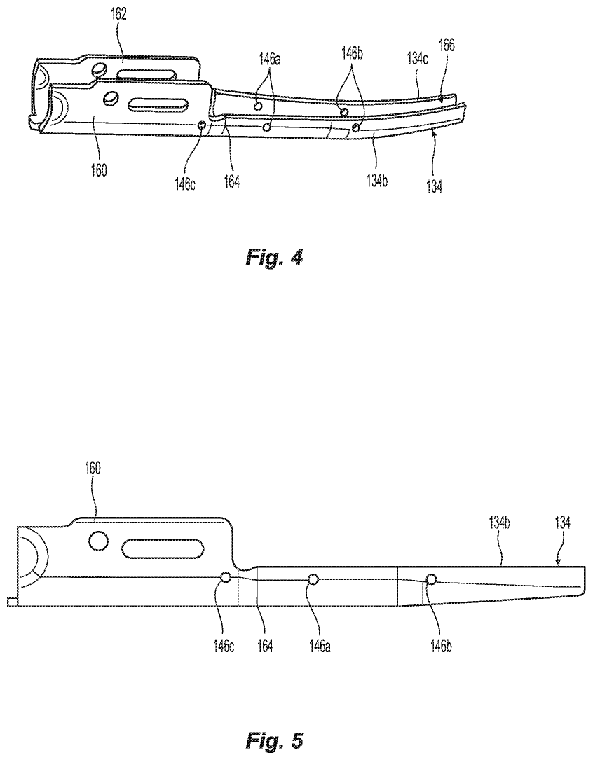

With reference to FIGS. 3, 6, and 7, the insulative housing 142 may be fabricated from a similar material as the jaw insert 136 and is overmolded around the lateral sides 134a, 134b of the jaw body 134 and the lateral sides 148a, 148b of the seal plate 148. The insulative housing 142 may pass under the lateral sides 148a, 148b of the seal plate 148 and between the jaw insert 136 and the jaw body 134. Consequently, the insulative housing 142 holds the remaining components of the jaw member 132 together, namely the seal plate 148, the jaw insert 136, and the jaw body 134. During manufacture, the insulative housing 142 may have protuberances that pass through the holes 146c (FIGS. 4 and 5) in the flanges 160, 162 and the holes 146a, 146b in the lateral sides 134a, 134b of the jaw body 134 to further anchor the insulative housing 142 to the jaw body 134.

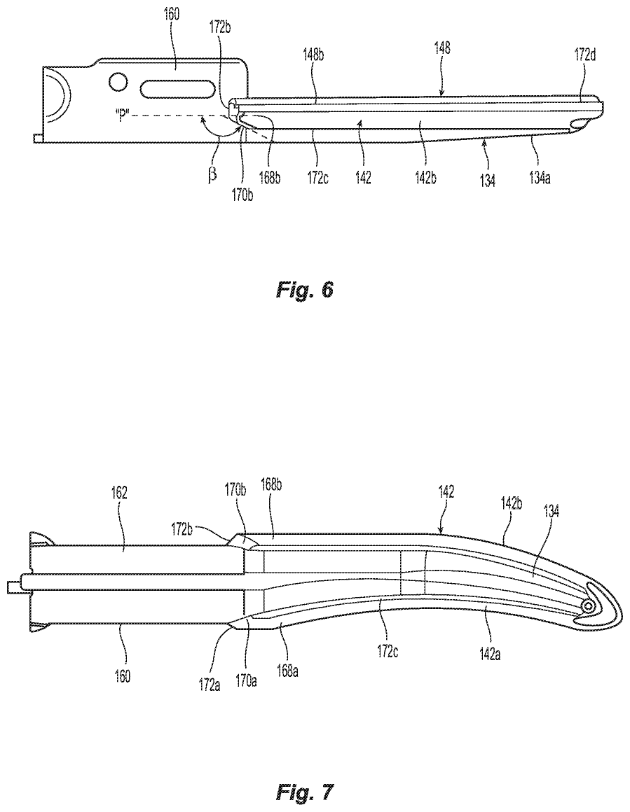

The insulative housing 142 has a pair of opposing lateral sides 142a, 142b attached to the respective pair of lateral sides 134a, 134b of the jaw body 134. Each of the lateral sides 142a, 142b of the insulative housing 142 has a proximal end 168a, 168b overlapping the respective holes 146c (FIGS. 4 and 5) formed in the pair of flanges 160, 162. The proximal end 168a, 168b of each of lateral sides 142a, 142b of the insulative housing 142 defines an oblique surface 170a, 170b. The oblique surfaces 170a, 170b each extend from a proximal edge 172a, 172b of the insulative housing 142 to a bottom edge 172c of the insulative housing 142. The proximal edges 172a, 172b of the insulative housing 142 are substantially perpendicular to a longitudinal plane “P” (FIG. 6) defined by the sealing plate 148. In some aspects, the proximal ends 168a, 168b of the insulative housing 142 may be devoid of the proximal edges 172a, 172b, such that the oblique surfaces 170a, 170b may extend directly from a top edge 172d of the insulative housing 142 to the bottom edge 172c.

In a distal direction, the oblique surfaces 170a, 170b of the insulative housing 142 extend downwardly at an angle β from the proximal edge 172a, 172b of the insulative housing 142 to the bottom edge 172c. The angle β of the oblique surfaces 170a, 170b is non-parallel with and non-perpendicular to the longitudinally-extending plane “P” defined by the sealing plate 148. For example, the angle β may be between about 5° and about 70° from the longitudinal plane “P.” In some aspects, the angle β of the oblique surfaces 170a, 170b may be between about 30° and 50° from the longitudinal plane “P.” In addition, as viewed from the bottom of the jaw member 132, as shown in FIG. 7, the lateral sides 142a, 142b of the insulative housing 142 are angled inwardly toward a central longitudinal axis defined by the jaw member 132. It is contemplated that the lateral sides 142a, 142b may be set at an angle of about 30° and 50° from a plane that is perpendicular to the longitudinal plane “P.”

During manufacture of the lower jaw member 132, the jaw insert 136 is received in the cavity 166 defined in the jaw body 134, and the sealing plate 148 is positioned on the jaw insert 136. Alternatively, the jaw insert 136 may be overmolded to the sealing plate 148 and the overmolded sub-assembly of the jaw insert 136 and sealing plate 148 subsequently positioned in the jaw body 134. The insulative housing 142 is then overmolded onto the lateral sides 134a, 134b of the jaw body 134, the lateral sides 148a, 148b of the sealing plate 148, and the lateral sides 136a, 136b of the jaw insert 136, thereby assembling the sealing plate 148, the jaw insert 136, and the jaw body 134 into one unit. The insulative housing 142 may be overmolded to the jaw body 134 in either a single-shot or a two-shot injection molding process such that the sealing plate 148 is coupled to and in spaced relation with the jaw body 134. Additionally or alternatively, the insulative housing 142 may be mechanically coupled to the jaw body 134 using tabs (not explicitly shown) that are received in the respective holes 146a-c of the flanges 160, 162 and the jaw body 134. Various other features may be molded into the insulative housing 142 that facilitate the attachment of the sealing plate 148 to the jaw insert 136. For example, ridges may be formed that permit ultrasonic welding of the sealing plate 148 onto the jaw insert 136.

With brief reference to FIG. 2A, the upper jaw member 130 includes the same major components as the lower jaw member 132, including a jaw body 143, a jaw insert (not explicitly shown), the sealing plate 150, and an insulative housing 151, and is constructed in a similar or same manner as the lower jaw member 132. The insulative housing 151 of the upper jaw member 130 has a proximal end 153 that terminates distally of the proximal edge 168a of the insulative housing 142 of the lower jaw member 132. Therefore, during removal of the end effector 114 from a trocar, the proximal edge 168a of the insulative housing 142 of the lower jaw member 132 contacts the trocar before the proximal edge 153 of the insulative housing 151 of the upper jaw member 130.

In use, the end effector 114 may be passed through a trocar into a surgical site. After or during performance of a surgical procedure, the end effector 114 may be removed from the surgical trocar. During removal of the end effector 114 from the trocar, the oblique surfaces 170a, 170b of the insulative housing 142 of the lower jaw member 132 may contact the trocar prior to the insulative housing 151 of the upper jaw member 130 due to the insulative housing 142 of the lower jaw member 132 extending proximally beyond the insulative housing 151 of the upper jaw member 130. Upon contacting the trocar, the oblique surfaces 170a, 170b assist in guiding the end effector 114 through the trocar and preventing the proximal end 153 of the insulative housing 151 of the upper jaw member 130 from catching on the trocar. Since holes 146c in respective flanges 160, 162 are disposed behind oblique surfaces 170a, 170b, there is added structural support at the oblique surfaces 170a, 170b to withstand rubbing against the trocar during removal of the end effector 114 from the trocar.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A jaw member of an electrosurgical forceps, comprising:
a jaw body;
an electrically conductive tissue sealing surface extending along a length of the jaw body and configured to connect to a source of electrosurgical energy for conducting electrosurgical energy through tissue; and
an insulative housing coupling the jaw body and the electrically conductive tissue sealing surface, wherein the insulative housing has a proximal end defining an oblique surface.

2. The jaw member according to claim 1, wherein the oblique surface terminates proximally at a proximal edge of the insulative housing.

3. The jaw member according to claim 2, wherein the proximal edge of the insulative housing extends perpendicularly relative to a longitudinal plane defined by the electrically conductive tissue sealing surface.

4. The jaw member according to claim 2, wherein the oblique surface extends downwardly at an angle from the proximal edge of the insulative housing to a bottom surface of the insulative housing.

5. The jaw member according to claim 4, wherein the oblique surface is non-parallel relative to a longitudinal plane defined by the electrically conductive tissue sealing surface and non-perpendicular relative to the longitudinal plane.

6. The jaw member according to claim 5, wherein the oblique surface is a pair of oblique surfaces each disposed on a respective lateral side of the insulative housing.

7. The jaw member according to claim 1, wherein the insulative housing is overmolded to the jaw body and the electrically conductive tissue sealing surface.

8. The jaw member according to claim 1, wherein the jaw body has a pair of lateral sides, the insulative housing overmolded to the pair of lateral sides.

9. The jaw member according to claim 8, wherein each of the pair of lateral sides of the jaw body defines a plurality of holes for receiving a portion of the insulative housing therein.

10. The jaw member according to claim 9, wherein at least one hole of the plurality of holes is disposed adjacent the oblique surface of the insulative housing.

11. The jaw member according to claim 9, further comprising a pair of laterally spaced parallel flanges extending proximally from a proximal end of the jaw body, wherein two holes of the plurality of holes is formed in the respective pair of flanges.

12. The jaw member according to claim 11, wherein the oblique surface is a pair of oblique surfaces each having at least a portion disposed over the respective pair of flanges.

13. The jaw member according to claim 9, wherein the plurality of holes is three holes.

14. The jaw member according to claim 1, further comprising a jaw insert disposed within the jaw body and electrically insulating the electrically conductive tissue sealing surface from the jaw body.
